(19) United States
(12) Patent Application Publication
(10) Pub. No.: US 2009/0221904 A1
(43) Pub. Date: Sep. 3, 2009

(54) INFLAMMATORY CONDITION PROGRESSION, DIAGNOSIS AND TREATMENT MONITORING METHODS, SYSTEMS, APPARATUS, AND USES

(76) Inventors: David J. Shealy, Downingtown, PA (US); Edmund Kwok, West Henrietta, NY (US); Steven Proulx, Baldwinsville, NY (US); Christopher T. Ritchlin, Canandaigua, NY (US); Edward M. Schwarz, Rochester, NY (US); Zhigang You, Rochester, NY (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/299,171

(22) PCT Filed: May 3, 2007
(86) PCT No.: PCT/US07/68091

§ 371 Date: Apr. 13, 2009

Related U.S. Application Data
(60) Provisional application No. 60/797,825, filed on May 4, 2006.

(51) Int. Cl. A61B 5/055; A61B 8/13

(52) U.S. Cl. 600/419; 600/437

(57) ABSTRACT

The present invention relates to at least one method, apparatus and/or system for providing at least one lymph node volume for use in the monitoring of progression, diagnosis or treatment of an inflammatory condition, as well as to a computer program product comprising software code portions for implementing the method in accordance with the invention.

### PRIORITY APPLICATION

This application claims priority to U.S. Provisional Application No. 60/797,825, filed May 4, 2006 and PCT/US07/68091 filed May 3, 2007, both applications are incorporated herein by reference.

### BACKGROUND OF THE INVENTION

1. Field of the Invention

The present invention relates to at least one method, apparatus and/or system for providing at least one lymph node volume for use in the monitoring of progression, diagnosis or treatment of an inflammatory condition, as well as to a computer program product comprising software code portions for implementing the method in accordance with the invention.

2. Related Art

A variety of different methods are currently used to diagnose the early stages of rheumatoid arthritis (RA), track its progression, and to monitor response to treatment. Most rheumatologists monitor RA using some or all of the 1987 American College of Rheumatology criteria (see, e.g., Arnett et al 1988, Arthritis Rheum. 31:315-324) which include morning stiffness, swollen/tender joints especially the hands, symmetric presentation, rheumatoid nodules, serum rheumatoid factor and radiographic changes; but these methods suffer from the problems of being subjective and/or not very sensitive for detection of early disease pathogenesis or for tracking changes in the rate of disease progression (see, e.g., Harle et al 2005, Rheumatology 44:426-433). While radiographs are useful in the clinical trial setting where the collection and analysis of data can be carefully controlled, this technique exposes the patient to ionizing radiation and images can be difficult to read and/or interpret. Other techniques include additional tests with samples of blood such as erythrocyte sedimentation rate and C-reactive protein (see, e.g., Ward 2003, J Rheumatol 31:884-895) or antibodies to cyclic citrullinated peptides (see, e.g., Reidemann et al 2005, Clin Exp Rheumatol 23:S69-76), none of which provide significant improvements over the previously mentioned criteria. Additional noninvasive tests such as ultrasound or magnetic resonance imaging of soft tissue are showing promise but are not yet routinely used (see, e.g., Ostergaard et al 2005, Best Pract Res Clin Rheumatol 19:91-116). There is a need for a diagnostic method that is simple, easy to analyze, preferably noninvasive and sensitive to changes in disease status due to progression or response to treatment.

### SUMMARY OF THE INVENTION

The present invention provides methods, systems and apparatus for early for monitoring, continued diagnosis, treatment effectiveness, and evaluation of arthritis and related inflammatory diseases, such as, but not limited to rheumatoid arthritis. Lymph node volume determined by imaging, such as but not limited to, magnetic resonance imaging, can be used as an early, noninvasive biomarker test to diagnose and monitor inflammatory disease activity and treatment, such as joint disease activity, or as a diagnostic test to follow inflammatory disease activity and treatment. Lymph node volume has been now discovered to directly correlate with, and/or is predictive of, inflammatory condition treatment effectiveness in reducing signs and symptoms of inflammatory conditions, such as joint inflammation and/or arthritis, such as, but not limited to rheumatoid arthritis and osteoarthritis, as well as other inflammatory and related conditions and subconditions.

The invention provides a non-invasive method for predicting or monitoring of inflammatory conditions in a human patient, comprising determining lymph node volume adjacent to a potentially inflamed area using a non-invasive imaging apparatus, wherein the extent or change in lymph node volume from a normal or non-inflammatory lymph node reference volume is predictive or indicative of inflammation in said potentially inflamed. The invention also provides wherein said inflammatory condition is selected from arthritis, systemic onset juvenile rheumatoid arthritis, psoriatic arthritis, ankylosing spondilitis, seronegative arthropathies, and osteoarthritis. The invention also provides wherein the arthritis is rheumatoid arthritis. The invention also provides wherein the potentially inflamed area is a joint. The invention also provides wherein the joint is selected from a knee joint, a shoulder joint, a hip joint, a finger joint, a toe joint, a wrist joint, an ankle joint, an elbow joint, a neck joint or a spinal joint.

The invention also provides wherein the imaging is by means of at least one of CT, CT-A, MRI, T1-MRI, MR-A, fMRI, PET, MEG, SPECT or ultrasound, and preferably by MRI.

The invention also provides wherein the, wherein said lymph node is associated with a pannus. The invention also provides wherein the lymph node is a popliteal lymph node.

The invention also provides wherein the method is used to predict said inflammatory condition or the location of said inflammatory condition. The invention also provides wherein the method is used to monitor treatment of said inflammatory condition. The invention also provides wherein the method is used to monitor disease progression of said inflammatory condition.

The invention also provides wherein the determination of lymph node volume is selected from visual inspection of image representation of said imaging, computer calculation of data set corresponding to lymph node image representation or computation of said imaging, and visual measurement of said imaging.

The invention also provides a system for non-invasive predicting or monitoring of inflammatory conditions in a human patient, comprising an imaging device for determining lymph node volume adjacent to a potentially inflamed area using a non-invasive imaging apparatus, wherein the extent or change in lymph node volume from a normal or non-inflammatory lymph node reference volume is predictive or indicative of inflammation in said potentially inflamed area. The invention also provides wherein the inflammatory condition is selected from arthritis, systemic onset juvenile rheumatoid arthritis, psoriatic arthritis, ankylosing spondilitis, seronegative arthropathies, and osteoarthritis. The invention also provides wherein the arthritis is rheumatoid arthritis.

The invention also provides wherein the potentially inflamed area is a joint. The invention also provides wherein the joint is selected from a knee joint, a shoulder joint, a hip joint, a finger joint, a toe joint, a wrist joint, an ankle joint, an elbow joint, a neck joint or a spinal joint.

The invention also provides wherein the apparatus for said imaging is by means of at least one of CT, CT-A, MRI, T1-MRI, MR-A, fMRI, PET, MEG, SPECT or ultrasound. V imaging is preferably by MRI.

The invention also provides wherein the lymph node is associated with a pannus. The invention also provides wherein the lymph node is a popliteal lymph node.

The invention also provides wherein the method is used to predict said inflammatory condition or the location of said inflammatory condition. The invention also provides wherein the method is used to monitor treatment of said inflammatory condition. The invention also provides wherein the system is used to monitor disease progression of said inflammatory condition.

The invention also provides wherein the determination of lymph node volume is selected from visual inspection of image representation of said imaging, computer calculation of data set corresponding to lymph node image representation or computation of said imaging, and visual measurement of said imaging.

In accordance with a first aspect of the present invention, a method for measuring lymph node volume for diagnosis, treatment or monitoring of inflammations is provided, in which a data set whose data values represent the lymph node volume is determined by imaging measurement and analysis and/or displayed two- or three-dimensionally, the method comprising computing a synthesized data set and/or synthesized representation from at least one selected diagnostic data sets that can be used to determine lymph node volume.

In principle, a plurality of different known imaging functions can be used to determine the lymph node volume. Examples of such mathematical functions are known from the related art, in connection with image processing or imaging. For example, a CT (computer tomography) method may be used for capturing a first selected data set, by which method x-ray diffracting structures can be particularly well resolved, and an MR (magnetic resonance) method may be used for capturing hydrogenous tissue structures can be particularly well captured. MR imaging or MRI is a preferred method of this invention.

In principle, more than one selected data set may also be synthesized into a data set in accordance with the invention, said data set providing the ability to determine relative lymph node volumes over time or for an initial diagnosis, through computer generated output as a numeric or graphical display, e.g., comparing relative lymph node size over time to show inflammatory disease progression.

Optimally displaying data sets graphically, which have been captured by methods of diagnosis, usually necessitates using various image display parameters. It is thus particularly advantageous for the image to be processed and displayed by means of preset parameters, tailored to the methods of diagnosis used in each case to capture a selected data set or to highlight certain tissue structures in a selected data set. In this way, the image information of the selected data set used in each case can be displayed particularly well, without any further computing or setting steps. It is particularly preferable to use at least one parameter for image processing or imaging, which influences the color and/or opacity allocation of the intensity values of the data sets. Image processing parameters are also known from the related art which influence other graphic properties of the data sets.

Preferably, the aforementioned parameters used for processing or displaying the image may also be determined manually or automatically. Expediently, processing and visualizing the image is initially undertaken by means of preset parameters, and the parameters are changed as required, for example when specific details of the three-dimensional visualization need to be highlighted in particular. For this purpose the parameters may be changed manually. The operator is able to recognize the imaging result by way of the display, and to change the parameters until the image display is expedient. In this arrangement, the imaging result may be visualized three-dimensionally, whereby the three-dimensional visualization can also preferably be rotated in three-dimensional space, or displayed as a predefined two-dimensional slice image through the lymph node volume for diagnosis of inflammatory conditions, such as rheumatoid arthritis, wherein the location of the slice through the lymph node volume for diagnosis of inflammatory conditions, such as rheumatoid arthritis may preferably be given, e.g. by the operator. In this way, the operator is able to directly affect visualization and optimize the parameters, in order to achieve optimal detail accuracy in visualization and optimal image information.

Particularly preferred for use in capturing data sets are the following methods: CT, CT-A, MRI, MR-A (magnetic resonance angiograph methodology), functional MRI or FMRI, PET (positron emission tomography), MEG (magnet encephalography), SPECT and ultrasound. However, the invention is not restricted to the aforementioned methods.

In accordance with a farther aspect, the present invention comprises a computer program product, directly loadable into the RAM of a digital computer and comprising software code portions for implementing the aforementioned steps in the method when the product is run on a computer. The computer program product may be stored on any data recording media, for example magnetic or magneto-optical disks, tapes, etc., or can be loaded via a network or the Internet. In particular preference, several computers can also use the computer program product at the same time.

In accordance with a farther aspect, the present invention comprises a system for determining the lymph node volume for diagnosis of inflammatory conditions, such as rheumatoid arthritis, including a data processing means for computing a synthesized data set, such that the data values of the synthesized data set are each computed as a mathematical function of at least one data value of each of the selected data sets, and also including a display for displaying the synthesized data set whose data values represent the lymph node volume for diagnosis of inflammatory conditions, such as rheumatoid arthritis.

A means may be provided for inputting the selected data sets into the data processing means. The input means may be a typical data interface with external data storage means, for loading buffered data sets into the system, or at least one input means may be coupled to a medical diagnosis apparatus, to capture a data set such that the system in accordance with the invention can then also be operated in real time.

The at least one selected data set may be selected by means of a menu control, for example manually by means of a computer program selecting the data sets on the basis of defined parameters, in particular automatically, or in some other way, as known in the art.

The system is preferably designed as a commercially available workstation, the aforementioned means preferably being realized in the form of software. The aforementioned steps in the method are also preferably realized in the form of software, or software modules or software code portions.

The synthesized data sets and/or the selected data sets and/or slice images obtained from the selected data sets are preferably displayed at predetermined points on a display, such that the operator has extensive image information and options for diagnosis at his disposal, in a compact form.

The system in accordance with the invention may also be realized as a module in a typical system for capturing data sets with the aid of an imaging method of diagnosis, for example in a computer tomography, whereby the other selected data set or sets can then be transferred from a data storage or a network.

The present invention further provides any invention described herein.

### DESCRIPTION OF THE INVENTION

The present invention provides methods, systems and apparatus for early for monitoring, continued diagnosis, treatment effectiveness, and evaluation of arthritis and related inflammatory diseases, such as, but not limited to rheumatoid arthritis. Lymph node volume determined by imaging, such as but not limited to, magnetic resonance imaging, can be used as an early, noninvasive biomarker test to diagnose and monitor inflammatory disease activity and treatment, such as joint disease activity, or as a diagnostic test to follow inflammatory disease activity and treatment. Lymph node volume has been now discovered to directly correlate with, and/or is predictive of, inflammatory condition treatment effectiveness in reducing signs and symptoms of inflammatory conditions, such as joint inflammation and/or arthritis, such as, but not limited to rheumatoid arthritis and osteoarthritis, as well as other inflammatory and related conditions and subconditions.

The present invention also provides a method for diagnosis and monitoring of treatment of at least one immune related disease, in a cell, tissue, organ, animal, or patient including, but not limited to, at least one of rheumatoid arthritis, juvenile rheumatoid arthritis, systemic onset juvenile rheumatoid arthritis, psoriatic arthritis, ankylosing spondilitis, gastric ulcer, seronegative arthropathies, osteoarthritis, and the like. See, e.g., the Merck Manual, 12th-17th Editions, Merck & Company, Rahway, N.J. (1972, 1977, 1982, 1987, 1992, 1999), Pharmacotherapy Handbook, Wells et al., eds., Second Edition, Appleton and Lange, Stamford, Conn. (1998, 2000), each entirely incorporated by reference.

Data sets corresponding to the lymph node image may be captured using a CT method (computer tomography), a CT method, a magnetic resonance method (MR), an MR angiograph method, a positron emission tomography method (PET), a functional MRI method (fMRI), an x-ray rotational angiograph method, a 3D ultrasound method, MEG (magnetic encephalography), or any other imaging method of medical diagnosis. The data sets inputted into the image composer may, however, also be derived from one and the same data set by differing methods of image preprocessing, especially for variously highlighting differing tissue structures by means of differing image parameters, each being used for a different selected data set. The optional layers of the data sets or their input data sets are typically organized in two-dimensional layers, wherein the sum of the 2D layers of each data set represents the lymph node volume to be displayed. For two-dimensional display, axial, sagittal or coronal slices through the lymph node volume are particularly suitable, although input data sets may also be organized differently.

Each data set can be stored in a data storage means and retrieved by the image composer, for example as selected by the operator. For this purpose, the composer is connected to the data storage means via an interface, a network or a comparable means. At least one of the data sets may, however, also can be captured in real time by a diagnostic device.

The image composer comprises a section for spatial allocation R, R′, an image combination section and at least one imaging section. Each of the sections is preferably implemented as software. Once selected by an operator or by a computer program running on the image composer, the image combination section combines or synthesizes at least two of the data sets in accordance with a definable image combination algorithm. This algorithm realizes a mathematical function which preferably assigns each new data value to the data values of the selected data sets with a corresponding spatial location on a one-to-one basis, as will be described in more detail below by way of an example. The sum of the data values computed in this way forms the synthesized data set. The mathematical function may also combine a number of respective data values of the selected data sets into a single data value of the synthesized data set with a corresponding spatial allocation or relationship. In the simplest case, adding and/or subtracting data values to/from each other of two selected data sets may be employed as the image combination algorithm, or also other image combination algorithms suitable for diagnostic visualization.

In order that the selected data sets may be superimposed with exact positioning, the spatial geometry of the selected data set, and also other parameters, such as for example the zoom factor of each data set, is taken into account, so that the data sets can be captured in various reference systems. Preferably, the selected data sets are spatially arranged precisely with respect to each other. The spatial allocation or relationship may be rigid, i.e. non-variable. As indicated by the broken line frames, the spatial allocation may also be elastic, i.e. variable, so that for example distortions occurring in a selected data set (for example in an MRI method) relative to a second selected data set 8 can be corrected prior to or during synthesizing. The spatial allocation R of the data values may be achieved prior to image pre-processing or thereafter.

The selected data sets are combined with each other by synthesizing the image information or image information derived there from, by suitable mathematical functions. In the image composer, at least one of the selected data sets can be subjected to 2D or 3D imaging or image processing, in order for example to highlight tissue structures in the data set particularly well. For medical diagnostic visualization methods, suitable image processing methods are known. Parameters are required for each of the image processing methods employed. These image-processing parameters can be predefined, or defined manually or automatically, as explained below.

Once synthesized, the synthesized data set can optionally be displayed in a two-dimensional slice display on the display unit 6, wherein location and orientation of the slice through the lymph node volume for diagnosis of inflammatory conditions, such as rheumatoid arthritis may be predefined, for example by a slider, a trackball or plus/minus buttons on a touch screen.

A three-dimensional visualization is also computed from the computed, synthesized data set, and displayed on the display unit. This visualization can be rotated in any way in three-dimensional space, for example by menu control, trackball or plus/minus buttons on a touch screen, wherein portions of the lymph node volume for diagnosis of inflammatory conditions, such as rheumatoid arthritis may be displayed enlarged or rotated.

The display shown on the display unit comprises image information from each of the selected data sets. For example, the image composer may select a CT image and/or an MR image. The CT image can in principle provide a particularly good resolution of part of the hard tissue structure. The magnetic resonance image (MR) in principle provides good resolution of the soft tissue structure, and where necessary of the vascular structure too, but not of the bone structure. The synthesized data set thus simultaneously comprises image information relating to the bone structure, the vascular structure and the brain structure. If a PET image is additionally selected, with which metabolically active areas in particular may be visualized, these areas may also be displayed in the synthesized data set. For synthesizing the data set, the selected data sets may be added, for example with predefined weighting or opacity and/or color rendering of the selected data sets, as will be described more exactly below.

For synthesizing the data set, each of in the simplest case two selected data sets may also be subtracted from one another. When, for example, a data set captured by means of an MR method is subtracted from a data set captured by an MR angiograph method, brain structures can be practically eliminated from the image, excepting the vascular structure. This may necessitate a suitable weighting of the respective selected data sets, or a suitable image processing of the selected data sets, as detailed below.

To enhance the information content of the synthesized data set, at least one of the selected data sets may be subjected to image processing to effectively highlight those structures contained in the selected data set which can be captured particularly well by the method used for capturing the selected data set. It is preferred to subject all selected data sets from image synthesis to image processing. For this purpose, preset parameters may be used which are known to be typically suitable for displaying data sets captured with the aid of the methods of diagnosis employed. However, the parameters may also be determined manually or automatically.

For methods of medical visualization, various parameters particularly suited to highlighting details in the display of images are known. These are typically parameters influencing the color and opacity assignment of the intensity values of the data sets. A few examples of preferred parameters are cited in the following. A threshold value, for example, may be set by the parameter, such that pixels whose value exceeds the threshold value are displayed bright and/or colored, and pixels whose data value does not reach the threshold value are displayed with a constant color or brightness, for example in black alone. A color and/or brightness gradient may also be influenced by the parameter, in order to scale the data values. The opacity or transparency of the image data values of a selected data set may also be influenced by the parameter, such that in a first data set displayed semi-transparent, three-dimensionally, a second set is recognizable. The parameter may also influence the color used to display a synthesized data set or a selected data set. Further image processing parameters are known from the related art.

To define the image processing parameter manually, a slice image is displayed by a selected data set on the display unit, wherein the three-dimensional location and orientation of the slice image may be predefined by means of operating elements. By means of a parameter setting device, one or more image processing parameters are modified until the slice image shown on the display unit or the three-dimensional display on the display unit exhibits the desired resolution and image information.

For implementing the method as described above, a computer program product is also disclosed, comprising software code portions for implementing the aforementioned steps in the method when the software code portions are loaded into the RAM of a digital computer. The syntheswized representation can, in accordance with the present invention, be displayed directly on a display, e.g. used directly for display control. A synthesized data set can, however, also be calculated which is displayed on a display after further processing (e.g. in a graphics card), intermediate storage, or the like. The present invention is not restricted to the methods of diagnosis cited above for capturing image data sets. In accordance with the present invention, any method of three-dimensional diagnostic visualization may be used, wherein each of the image data sets may be composed and processed in any way, for synthesizing the synthesized data set.

### EXAMPLE 1

### Use of MRI to Diagnose and Monitor Treatment of Arthritis Using Popliteal Lymph Node Volume.

Transgenic mice that constitutively express human TNF develop arthritis with joint degradation that is similar to rheumatoid arthritis. TNF transgenic mice (5-6 months old) were treated with anti-human TNF or placebo (N=5). 3T MRI (Siemens) was performed at baseline and every four weeks using a custom mouse knee coil and T1 weighted scans (VirtualScopics) before and after gadolinium-DTPA injection (OmniScan). OsiriX quantified normalized bone marrow intensity (NBMI) and measured the marrow contrast enhancement (CE) after intravenous injection. Amira 3.1 was used for 3D reconstruction and quantification of popliteal lymph node and synovial volumes.

3D MRI demonstrated predicted changes with significance (p<0.05) for all biomarkers. The lymph node volume proved to be the most sensitive biomarker, as anti-TNF treatment resulted in a 57% decrease after 4 weeks. The placebo group progressed 311% in 8 weeks, and there was >10-fold difference between the groups at this time that was sustained through the rest of the study. There was also a 3-fold difference in pannus volume (placebo vs. anti-TNF) at 12 weeks, at 16 weeks this difference was reduced to 2.6× due to tissue necrosis. NBMI showed a significant decrease by 16 weeks in the anti-TNF group, but did not change in the placebo group. Finally, in mice treatment with placebo the CE values showed a significant increase at 12 weeks, however at 16 weeks this difference was no longer significant, again probably due to tissue necrosis effects on vascularity.

Advantages: This technique can be used as an objective measure to evaluate the progression of inflammatory arthritis and the efficacy of various treatments. The changes in lymph node volume appear to be a very early event that precedes joint inflammation, as determined by the pannus volume. Previous technologies that are currently used include radiographs or various blood tests such as sedimentation rate. The X-rays used to produce radiographs expose the patient to radiation, provide only a planar view and are difficult to read. Sedimentation rate is an indirect measure of disease activity and requires drawing a blood sample. This is the first longitudinal outcome measure for the onset and progression of inflammatory arthritis. It can predict which joints will develop inflammatory arthritis, when and how severe.

### EXAMPLE 2

### 3D-MRI Quantification of the Progression and Amelioration of Inflammatory Arthritis in Mice

Purpose. A limitation of mouse models of arthritis is the absence of a quantitative, longitudinal and translational outcome measure. Pre-clinical studies remain overly dependent on sacrificial outcomes that cannot faithfully evaluate pre-existing disease. To overcome this obstacle MRI was employed to track four biomarkers in TNF transgenic mice treated with anti-TNF therapy vs. placebo for 16 weeks.

Methods: TNF-Tg mice (5-6 months old) were treated with anti-TNF or placebo (N=5). 3T MRI was performed at baseline and every four weeks using a custom mouse knee coil and T1 weighted scans (VirtualScopics) before and after Gadolinium-DTPA injection. OsiriX quantified normalized bone marrow intensity (NBMI) and measured the marrow contrast enhancement (CE) after i.v. injection. Amira 3.1 was used for 3D reconstruction and quantification of popliteal lymph node and synovial volumes.

Results 3D MRI demonstrated predicted changes with significance (p<0.05) for all biomarkers. The lymph node volume proved to be the most sensitive biomarker, as anti-TNF treatment resulted in a 57% decrease after 4 weeks. The placebo group progressed 311% in 8 weeks, and there was >10-fold difference between the groups at this time that was sustained through the rest of the study. There was also a 3-fold difference in pannus volume (placebo vs. anti-TNF) at 12 weeks. At 16 weeks this difference was reduced to 2.6× due to tissue necrosis. NBMI showed a significant decrease by 16 weeks in the anti-TNF group, but did not change in the placebo group. Finally, in mice given placebo the CE values showed a significant increase at 12 weeks, however at 16 weeks this difference was no longer significant, again probably due to tissue necrosis effects on vascularity.

Conclusions 3D MRI can be used to sensitively detect serial changes in biomarkers associated with inflammatory arthritis in murine models. By using a well-established model (TNF-Tg) and proven anti-TNF therapy we were able to validate 4 independent biomarkers of inflammatory arthritis and demonstrate significant changes within 4 weeks. We also find that massive tissue necrosis limits the linear progression of inflammatory arthritis in this model, such that long-term studies are limited by this endpoint.

It will be clear that the invention can be practiced otherwise than as particularly described in the foregoing description and examples.

Numerous modifications and variations of the present invention are possible in light of the above teachings and, therefore, are within the scope of the appended claims.

What is claimed is:

1. A non-invasive method for predicting or monitoring of inflammatory conditions in a human patient, comprising determining lymph node volume adjacent to a potentially inflamed area using a non-invasive imaging apparatus, wherein the extent or change in lymph node volume from a normal or non-inflammatory lymph node reference volume is predictive or indicative of inflammation in said potentially inflamed area.

2. The method of claim 1, wherein said inflammatory condition is selected from arthritis, systemic onset juvenile rheumatoid arthritis, psoriatic arthritis, ankylosing spondilitis, seronegative arthropathies, and osteoarthritis.

3. The method of claim 2, wherein said arthritis is rheumatoid arthritis.

4. The method of claim 1, wherein said potentially inflamed area is a joint.

5. The method of claim 1, wherein said joint is selected from a knee joint, a shoulder joint, a hip joint, a finger joint, a toe joint, a wrist joint, an ankle joint, an elbow joint, a neck joint or a spinal joint.

6. The method of claim 1, wherein said imaging is by means of at least one of CT, CT-A, MRI, T1-MRI, MR-A, fMRI, PET, MEG, SPECT or ultrasound.

7. The method of claim 5, wherein said imaging is by MRI.

8. The method of claim 1, wherein said lymph node is associated with a pannus.

9. The method of claim 8, wherein said lymph node is a popliteal lymph node.

10. The method of claim 1, wherein said method is used to predict said inflammatory condition or the location of said inflammatory condition.

11. The method of claim 1, wherein said method is used to monitor treatment of said inflammatory condition.

12. The method of claim 1, wherein said method is used to monitor disease progression of said inflammatory condition.

13. The method of claim 1, wherein said determination of lymph node volume is selected from visual inspection of image representation of said imaging, computer calculation of data set corresponding to lymph node image representation or computation of said imaging, and visual measurement of said imaging.

14. A system for non-invasive predicting or monitoring of inflammatory conditions in a human patient, comprising an imaging device for determining lymph node volume adjacent to a potentially inflamed area using a non-invasive imaging apparatus, wherein the extent or change in lymph node volume from a normal or non-inflammatory lymph node reference volume is predictive or indicative of inflammation in said potentially inflamed area.

15. The system of claim 14, wherein said inflammatory condition is selected from arthritis, systemic onset juvenile rheumatoid arthritis, psoriatic arthritis, ankylosing spondilitis, seronegative arthropathies, and osteoarthritis.

16. The system of claim 15, wherein said arthritis is rheumatoid arthritis.

17. The system of claim 14, wherein said potentially inflamed area is a joint.

18. The system of claim 14, wherein said joint is selected from a knee joint, a shoulder joint, a hip joint, a finger joint, a toe joint, a wrist joint, an ankle joint, an elbow joint, a neck joint or a spinal joint.

19. The method of claim 1, wherein said apparatus for said imaging is by means of at least one of CT, CT-A, MRI, T1-MRI, MR-A, fMRI, PET, MEG, SPECT or ultrasound.

20. The system of claim 19, wherein said imaging is by MRI.

21. The system of claim 14, wherein said lymph node is associated with a pannus.

22. The system of claim 21, wherein said lymph node is a popliteal lymph node.

23. The system of claim 23, wherein said system is used to predict said inflammatory condition or the location of said inflammatory condition.

24. The system of claim 14, wherein said system is used to monitor treatment of said inflammatory condition.

25. The system of claim 14, wherein said system is used to monitor disease progression of said inflammatory condition.

26. The system of claim 14, wherein said determination of lymph node volume is selected from visual inspection of image representation of said imaging, computer calculation of data set corresponding to lymph node image representation or computation of said imaging, and visual measurement of said imaging.
